(19) United States
(12) Patent Application Publication
(10) Pub. No.: US 2006/0217675 A1
(43) Pub. Date: Sep. 28, 2006

(54) Diaper with diaper changing necessities

(76) Inventor: Maria L. Coelho, Bethel, CT (US)

Correspondence Address: JAMES RAY & ASSOCIATES, 2640 PITCAIRN ROAD, MONROEVILLE, PA, 15146, US

(21) Appl. No.: 11/301,719

(22) Filed: Dec. 13, 2005

Related U.S. Application Data
(60) Provisional application No. 60/664,910, filed on Mar. 24, 2005.

(51) Int. Cl. A61F 13/15

(52) U.S. Cl. 604/385.06; 604/385.19

(57) ABSTRACT

An article of manufacture includes a disposable diaper having at least one pocket which is formed intermediate interior and exterior layers of the diaper and which is provided with an elongated slit for accessing basic diaper necessities such as moist toilette, ointment and powder releaseably placed within the at least one pocket prior to providing the diaper for sale.

### CROSS-REFERENCE TO RELATED APPLICATIONS

This application is related to and claims priory from a Provisional Patent Application Ser. No. 60/664,910 filed Dec. 14, 2004.

### FIELD OF THE INVENTION

The present invention relates, in general, to diapers and, more particularly, this invention relates to disposable diapers having storage pockets containing at least one article of personal hygiene used during diaper change.

### BACKGROUND OF THE INVENTION

Disposable diapers have been utilized extensively by children and adults to protect garments from inadvertent release of bodily waste products. During diaper change, basic necessities of personal hygiene such as moist toilettes are used to clean soiled body portions and ointments and powders are used to treat body areas that developed or may develop diaper rash. As is generally well known, such moist toilettes, ointments and powders are purchased separately from disposable diapers and must be packed separately from disposable diapers for changes thereof outside of a home environment. Such situation increases effort in preparing for diaper changes outside of home environment and may cause frustration when some of the basic diaper changing necessities are not readily available.

Efforts have been made to make a process of diaper changes more convenient. U.S. Pat. No. 6,454,748 to Ives, G.B. Pat. No. 2298354 to French and U.S. Pat. Application Publication No. 2003/0109841 disclose disposable diapers having at least one pocket for storing basic diaper changing necessities.

However, the disadvantage of such prior art is that basic diaper changing necessities must be purchased separately and placed into the storage pocket.

### SUMMARY OF THE INVENTION

The present invention provides an article of manufacture which includes a disposable diaper having at least one pocket at least engageable with an exterior layer of the diaper. The at least one pocket is provided with an access element. At least one basic diaper change necessity such as moist toilette, ointment and powder is releaseably placed into the at least one pocket.

### OBJECTS OF THE INVENTION

It is, therefore, one of the primary objects of the present invention to provide a disposable diaper.

Another object of the present invention is to provide a disposable diaper which has a storage pocket formed therein.

Yet another object of the present invention is to provide a disposable diaper which has basic diaper changing necessities disposed in such storage pocket.

A further object of the present invention is to provide a disposable diaper with a storage pocket which has a concealed pocket access to deter attention of a child or an adult.

Yet a further object of the present invention is to provide a disposable diaper with a simple access to a storage pocket formed therein.

In addition to the several objects and advantages of the present invention which have been described with some degree of specificity above, various other objects and advantages of the invention will become more readily apparent to those persons who are skilled in the relevant art, particularly, when such description is taken in conjunction with the attached drawing Figures and with the appended claims.

### BRIEF DESCRIPTION OF THE DRAWINGS

FIG. 1 is a perspective view of the diaper of the invention particularly showing the pocket construction of the presently preferred embodiment;

FIG. 2 is a partial side view particularly illustrating alternative pocket construction.

FIG. 3 is a partial side view particularly illustrating pocket closure means of one alternative embodiment of the invention;

FIG. 4 is a partial plan view of the invention particularly illustrating pocket closure means of another alternative embodiment of the invention; and

FIG. 5 is a partial plan view of the invention particularly showing indicia disposed adjacent the pocket access.

### BRIEF DESCRIPTION OF THE VARIOUS EMBODIMENTS OF THE INVENTION

Prior to proceeding to the more detailed description of the present invention, it should be noted that, for the sake of clarity and understanding, identical components which have identical functions have been identified with identical reference numerals throughout the several views illustrated in the drawing figures.

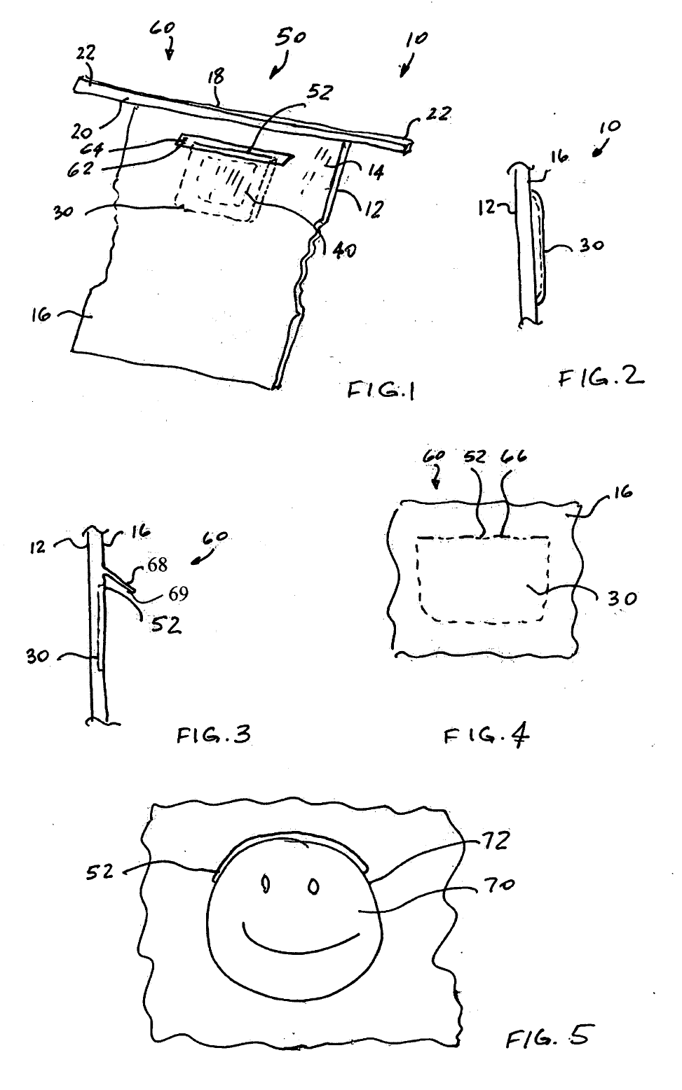

Reference is now made, to FIGS. 1-5, wherein there is shown a diaper, generally designated 10, of a well known disposable design. In a particular reference to FIG. 1, such diaper 10 includes an interior layer 12 of a liquid-previous material, an exterior layer 16 of a liquid-impervious material, and an absorbent core 14 formed intermediate the interior layer 12 and the exterior layer 16. A waste band 20 terminates at an upper edge 18 of the diaper 10 and has a pair of closing flaps 22 for securing such diaper 10 onto a wearer's body.

The diaper 10 further includes at least one pocket 30 at least engageable with the exterior layer 16 for holding at least one basic diaper change necessity member 40, such as a moist toilette, ointment and powder, which is inserted into the at least one pocket 30 prior to purchase of such diaper 10 by a consumer. It will be appreciated that a plurality of pockets 30 may be provided, each containing one basic diaper change necessity member 40. Such at least one basic diaper change necessity member 40 is disposed within the at least one pocket 30 so as to substantially reduce discomfort to a wearer of the diaper 10.

Access means, generally designated 50, is engageable with the at least one pocket 30 for removing such at least one basic diaper change necessity member 40 from the at least one pocket 30.

In the presently preferred embodiment of the invention, the at least one pocket 30 is concealed from view by being formed intermediate the exterior layer 16 and the interior layer 12 of the diaper 10. Such at least one pocket 30 may be of a liquid-impervious material for direct contact with the basic diaper change necessity member 40. Alternatively, each of the moist toilette, ointment and powder is prepackaged enabling the at least one pocket 30 to be formed from any liquid-previous material.

According to an alternative embodiment of the invention, the at least one pocket 30 is attached to the exterior layer 16 external to the diaper 10, as best shown in FIG. 2.

The presently preferred access means 50 is an elongated slit 52 having a predetermined shape. Preferably, such elongated slit 52 is straight and is disposed immediately below an upper edge of the diaper. The alternative shape of such elongated slit 52 is a curve.

Diaper 10 further includes a removable closure means, generally designated 60, for closing the access means 50. The presently preferred closure means 60, best shown in FIG. 1, is a strip of a transparent tape 62 applied to the exterior layer 16 of the diaper with an adhesive means 64 for sealing the elongated slit 52.

It will be appreciated that the combination of the elongated slit 52 and such strip of a transparent tape 62 is advantageous in concealing access to the at least one pocket 30 and deterring attention of a child or an adult to presence of such at least one pocket 30.

Now in reference to FIG. 3, therein shown an alternative closure means 60 which may be a flap 68 hingeably attached to the exterior surface of the diaper 10 or to the exterior surface of the externally attached at least one pocket 30. Such flap 68 may be of a self-closing type and may further include a retaining means 69, such as adhesive, zipper, Ziploc™ fastener, and Velcro™ hook-and-loop type fastener or button, for positively retaining the flap 68 over the access means 50.

According to yet another alternative embodiment, shown in FIG. 4, the closure means 60 may include a predetermined plurality of perforations 66 disposed in a predetermined pattern for closing elongated slot 52. The perforations 66 may be separated by pulling portions of the exterior layers 16 disposed on either side of such perforations 66 in opposite directions to gain access into the at least one pocket 30. Such predetermined pattern may be one of straight and curved.

Diaper 10 may be provided with predetermined indicia 70, disposed adjacent the access means 50 to the at least one pocket 30. The shape of a portion of a peripheral edge 72 of such indicia 70 may be identical to the shape of the access means 50, particularly being an elongated slit 52. Advantageously, a plurality of same indicia 70 having a smaller size may be provided on the waste band 20.

Thus, the present invention has been described in such full, clear, concise and exact terms as to enable any person skilled in the art to which it pertains to make and use the same. It will be understood that variations, modifications, equivalents and substitutions for components of the specifically described embodiments of the invention may be made by those skilled in the art without departing from the spirit and scope of the invention as set forth in the appended claims.

I claim:

1. An article of manufacture comprising:
(a) a diaper having at least one pocket at least engageable with an exterior layer of said diaper and access means engageable with said at least one pocket; and
(b) at least one of moist toilette, ointment and powder releaseably disposed within said at least one pocket.

2. The article of manufacture according to claim 1, wherein said at least one of moist toilette, ointment and powder is prepackaged.

3. The article of manufacture according to claim 1, wherein said at least one pocket is formed intermediate said exterior layer and an interior layer of said diaper.

4. The article of manufacture according to claim 1, wherein said at least one pocket is attached to an outer surface of said exterior layer.

5. The article of manufacture according to claim 1, wherein said access means is an elongated slit having a predetermined shape.

6. The article of manufacture according to claim 5, wherein said elongated slit is disposed immediately below an upper edge of said diaper.

7. The article of manufacture according to claim 5, wherein said predetermined shape is one of straight and curved.

8. The article of manufacture according to claim 1, wherein said diaper includes a closure means for covering said access means to said at least one pocket.

9. The article of manufacture according to claim 8, wherein said closure means is a strip of a transparent tape applied with an adhesive means to one of an outer surface of said exterior layer when said at least one pocket is formed intermediate said exterior layer and an interior layer of said diaper and to an exterior surface of said at least one pocket attached to an outer surface of said exterior layer for sealing said access means.

10. The article of manufacture according to claim 8, wherein said closure means is one of a zipper and Ziploc™ fastener.

11. The article of manufacture according to claim 8, wherein said closure means is a flap hingeably attached to said exterior layer when said at least one pocket is formed intermediate said exterior layer and an interior layer of said diaper and to an exterior surface of said at least one pocket attached to an outer surface of said exterior layer.

12. The article of manufacture according to claim 11, wherein said flap is self-closing.

13. The article of manufacture according to claim 11, wherein said article of manufacture includes a retaining means for positively retaining said flap over said access means.

14. The article of manufacture according to claim 11, wherein said retaining means is one of adhesive, zipper, Ziploc™ fastener, a Velcro™ hook-and-loop type fastener and button.

15. The article of manufacture according to claim 8, wherein said closure means is a predetermined plurality of perforations disposed in a predetermined pattern.

16. The article of manufacture according to claim 15, wherein said predetermined pattern is one of a straight and curved.

17. The article of manufacture according to claim 1, wherein said at least one pocket is formed from a liquid-impervious material.

18. The article of manufacture according to claim 1, wherein said diaper is disposable.
